(19) United States
(12) Patent Application Publication
(10) Pub. No.: US 2025/0099197 A1
(43) Pub. Date: Mar. 27, 2025

(54) Method and Apparatus For Patient Surgical Table

(71) Applicant: Dental Health Products, Inc., New Franklin, WI (US)

(72) Inventor: Scott Perry, Farmers Branch, TX (US)

(21) Appl. No.: 18/372,253

(22) Filed: Sep. 25, 2023

Related U.S. Application Data
(60) Provisional application No. 63/411,205, filed on Sep. 29, 2022.

(51) Int. Cl. A61B 50/15; A61G 15/14

(52) U.S. Cl. CPC A61B 50/15; A61G 15/14

(57) ABSTRACT

A novel over the patient adjustable surgical table that comprises a table top having a back rail, a crank, opposed telescoping vertical legs each attached to a base with the base provided with rolling coasters having safety brakes. With the crank and opposed telescoping vertical legs coacting to raise and lower the table, as needed, this creates a first clearance between the opposed telescoping vertical legs under the table top and within the spacing to accommodate the patient surgical chair or bed and a second clearance within the spacing between the table top and the patient lying on the surgical chair or bed enabling the surgeon to use the table situated over the patient to thereby perform surgery on the patient lying on the surgical chair or bed

### I. CROSS-REFERENCE TO RELATED APPLICATION

This patent application is a non-provisional application claiming priority from U.S. Provisional Patent Application Ser. No. 63/411,205, entitled “Method and Apparatus For Patient Surgical Table”, filed on Sep. 29, 2022, and is fully incorporated herein by reference.

### II. FIELD OF THE INVENTION

The present invention relates to patient surgical tables and, more particularly, to a novel adjustable patient surgical table that provides improved benefits to the user compared to currently available tables.

### III. DESCRIPTION OF THE PRIOR ART

The following identified patents, listed below, were revealed relative to moveable or adjustable medical tables:

| | Issued/ | |
|---|---|---|
| Patent No. | Published | Title of Patent/Application |
| JP5738012B2 | Jun. 17, 2015 | Caster Lock Device of Vertically |
| | | Moveable Table |
| JP3707689B2 | Oct. 19, 2005 | Table Apparatus |
| CN208525321U | Feb. 22, 2019 | Medical Lift Table |
| CN211354397U | Aug. 28, 2020 | Multifunctional Resting Table For |
| | | Seating Chronic Obstructive Pulmonary |
| | | Disease Patient |
| DE3347296A1 | Jul. 11, 1985 | Vertical Adjustment Assembly In |
| | | Particular For Tables |

Each of these prior art issued patents or published patent applications uncovered discloses the use of a table that can be moved up and down, such as an overbed or a bedside table for a hospital bed or the like. However, each of these devices in the prior art patents disclose a method or system that is limited in its application and/or different than Applicant's invention or device, which has solved problem presented for over the patient adjustable surgical tables.

Thus, there is a need, therefore, and there has never been disclosed Applicant's unique method and apparatus for over the patient adjustable surgical table.

### IV. SUMMARY OF THE INVENTION

The present invention is directed to a novel over the patient adjustable surgical table that comprises a table top having a back rail, a crank, opposed telescoping vertical legs each attached to a base with the base provided with rolling coasters having safety brakes. With the crank and opposed telescoping vertical legs coacting to raise and lower the table, as needed, this creates a first clearance between the opposed telescoping vertical legs under the table top and within the spacing to accommodate the patient surgical chair or bed and a second clearance within the spacing between the table top and the patient lying on the surgical chair or bed enabling the surgeon to use the table situated over the patient to thereby perform surgery on the patient lying on the surgical chair or bed.

### V. BRIEF DESCRIPTION OF THE DRAWINGS

The Description of the Preferred Embodiment will be better understood with reference to the following figures:

FIG. 1 is a perspective view of Applicant's inventive over the patient adjustable surgical table in accordance with the present invention and, in particular, illustrating the table in the fully lowered position.

FIG. 2 is a front view of Applicant's inventive over the patient adjustable surgical table in accordance with the present invention.

FIG. 3 is a back view of Applicant's inventive over the patient adjustable surgical table in accordance with the present invention.

FIG. 4 is a left side view of Applicant's inventive over the patient adjustable surgical table and, in particular, illustrating the table in the fully raised position.

FIG. 5 is a right side view of Applicant's inventive over the patient adjustable surgical table in accordance with the present invention.

FIG. 6 is a top view of Applicant's inventive over the patient adjustable surgical table in accordance with the present invention.

FIG. 7 is a bottom view of Applicant's inventive over the patient adjustable surgical table in accordance with the present invention.

FIG. 8 is a front view of Applicant's inventive over the patient adjustable surgical table in accordance with the present invention and, in particular, illustrating the table in the fully raised position.

FIG. 9 is a left side view of Applicant's inventive over the patient adjustable surgical table in accordance with the present invention and, in particular, illustrating the table in the fully raised position.

FIG. 10 is a perspective view of Applicant's inventive over the patient adjustable surgical table and, in particular, illustrating the surgical table accommodating a patient surgical chair or bed in accordance with the present invention.

FIG. 11 is a side view of Applicant's inventive over the patient adjustable surgical table and, in particular, illustrating the various external and internal components, including for the raising and lowering, of Applicant's inventive over the patient adjustable surgical table; and further illustrating the surgical table in the fully lowered position.

FIG. 12 is a side view of Applicant's inventive over the patient adjustable surgical table and, in particular, illustrating the various external and internal components, including for the raising and lowering, of Applicant's inventive over the patient adjustable surgical table; and further illustrating the surgical table in the fully raised position.

FIG. 13 is a front view of Applicant's inventive over the patient adjustable surgical table and, in particular, illustrating the various external and internal components, including for the raising and lowering, of Applicant's inventive over the patient adjustable surgical table; and further illustrating the surgical table in the fully lowered position.

FIG. 14 is a perspective view of Applicant's inventive over the patient adjustable surgical table, with portions removed, and, in particular, illustrating the various external and internal components, including for the raising and lowering, of Applicant's inventive over the patient adjustable surgical table.

### VI. DETAILED DESCRIPTION OF THE PREFERRED EMBODIMENT

While this invention is susceptible of embodiment in many different forms, there is shown in the drawings and described herein in detail specific embodiments with the understanding that the present disclosure is to be considered as an exemplification of the principles of the invention and is not intended to limit the invention to the embodiments illustrated.

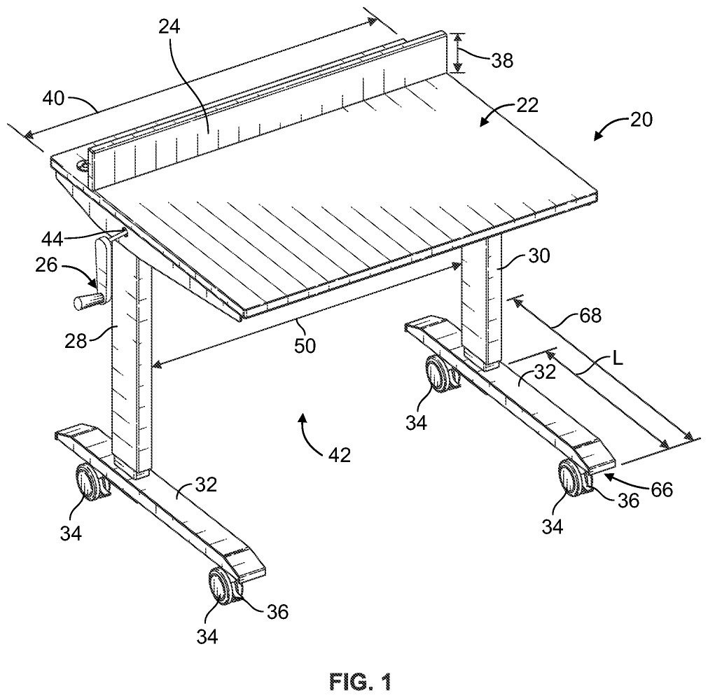

Turning to FIG. 1, there is illustrated Applicant's unique over the patient adjustable surgical table 20. In the preferred embodiment, the table 20 comprises a table top having a back rail 24, a crank 26, opposed telescoping vertical legs 28 and 30 each attached to a base 32, with each base 32 provided with rolling coasters 34 having safety brakes 36.

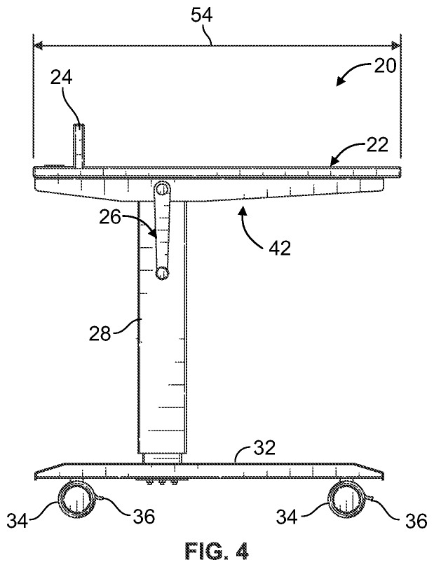

In the preferred embodiment, the table 20 is preferably made with King MediGrade® Antimicrobial Polymer material, any other antimicrobial material, and/or any other medical grade materials known to one skilled in the art. In the preferred embodiment, the table top 22 has a length 40 of approximately 42 inches and a width 54 (see FIG. 4) of approximately 27 inches.

The back rail 24 is situated in a vertical orientation to the table top 22 having a height 38 and the same length 40 extending across the entirety of the table top 22 to keep, for example, to keep instruments from rolling off the table top 22 when in use by a surgeon. In the preferred embodiment, the height 38 of the back rail 24 is approximately 3 inches.

The table top 22 and opposed telescoping vertical legs 28 and 30 create an spacing 42 under the table top 22 and between the opposed telescoping vertical legs 28 and 30.

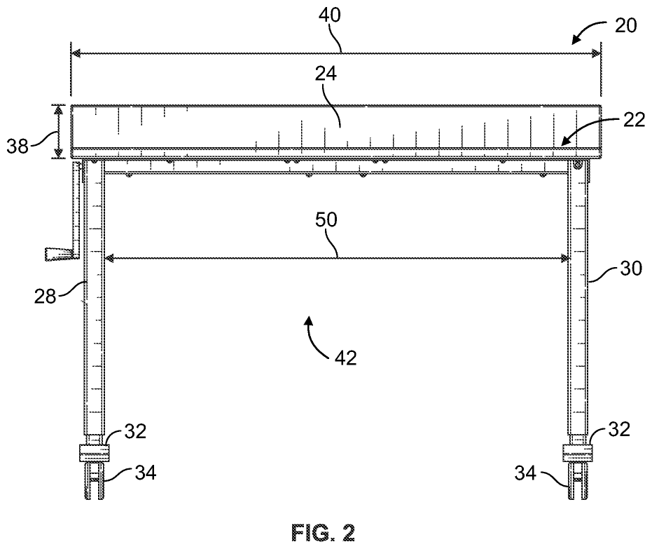

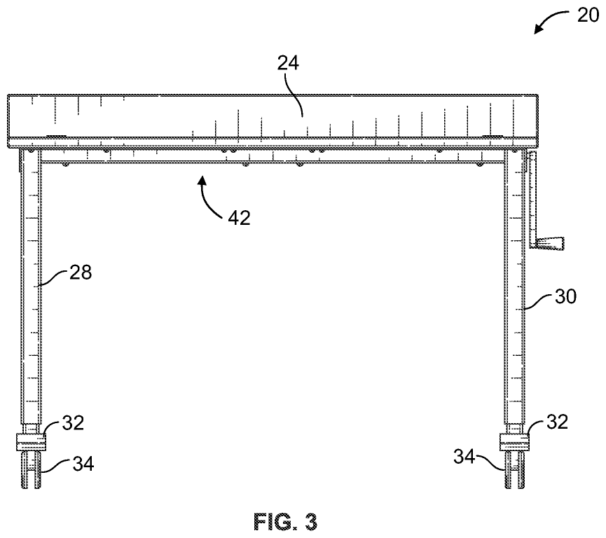

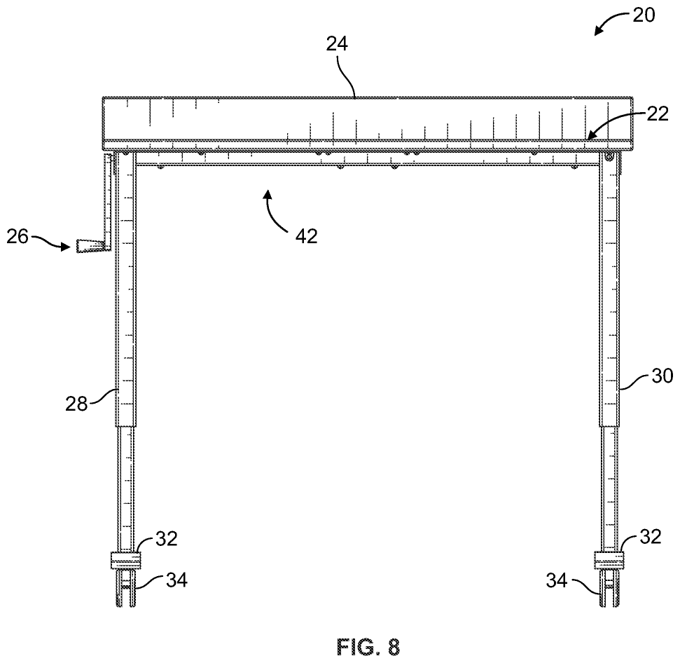

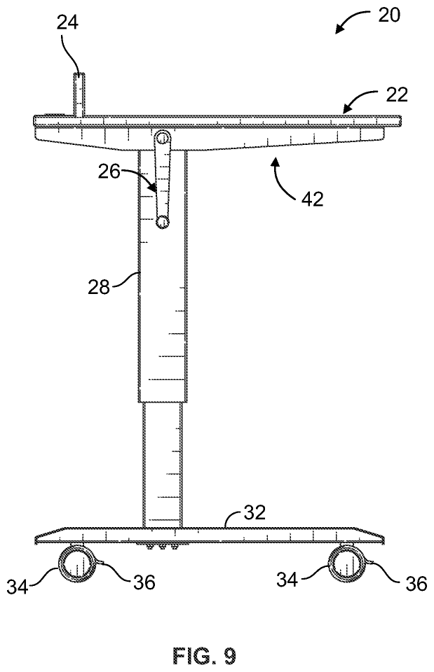

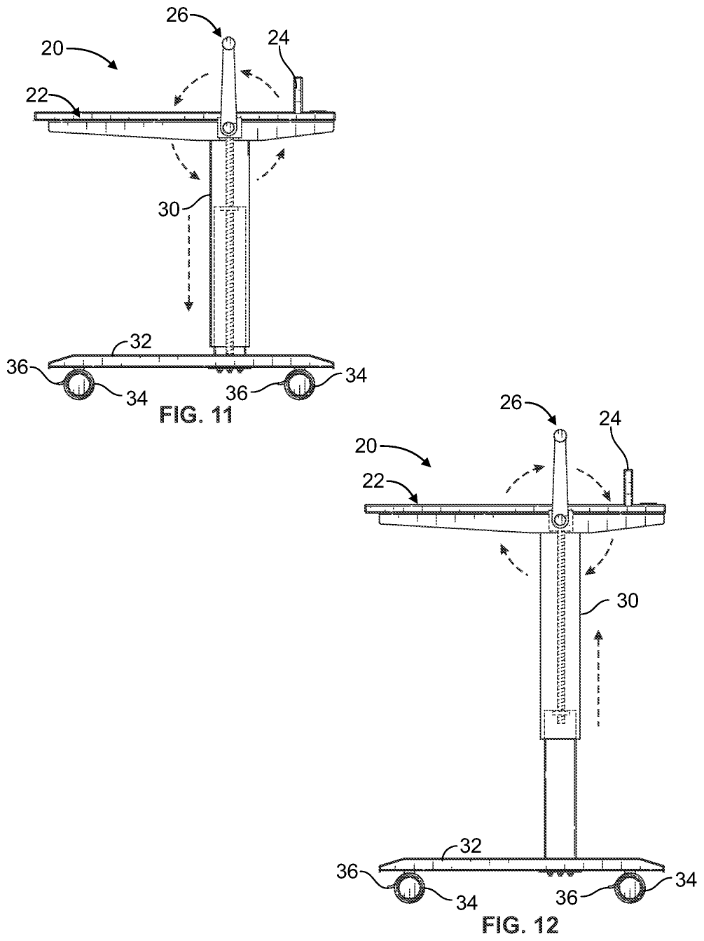

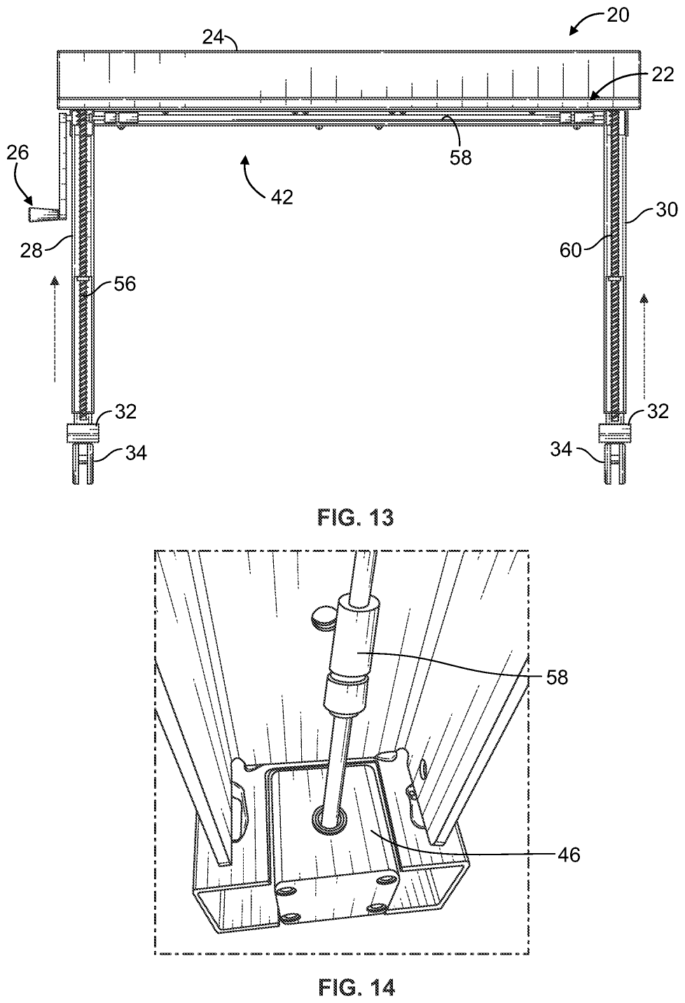

The crank 26 is provided for raising and lowering the opposed telescoping vertical legs 28 and 30 of the table 20. In the preferred embodiment, the crank 26 is connected to a rod 44 which is inserted into a sprocket 46 within the table top 22 (see FIG. 14). In the preferred embodiment, the sprocket 46 (a) is connected to a first chain drive or internal nut screw/jack screw (“chain drive”) 56 such that, upon rotation of the crank 26, engages the chain drive 56 which raises the telescoping vertical leg 28; and (b) is connected to an elongated arm 58, situated and extending under the table top 22, which is further connected to a second chain drive or internal nut screw/jack screw (“chain drive”) 60 such that, upon the same rotation of the crank 26, the elongated arm 58 is rotated which in turn engages the chain drive 56 which raises the telescoping vertical leg 30 to, collectively, raise the table 20 into a fully raised position, as illustrated in FIGS. 8, 9, and 12, or lower the table 20 into a fully lowered position, as illustrated in FIGS. 1-5, and 11. In this manner, both the opposed telescoping vertical legs 28 and 30 are raised and lowered simultaneously using the same crank 26. Alternatively, the opposed telescoping vertical legs 28 and 30 could be raised and lowered simultaneously using separate cranks 26.

In the preferred embodiment, the table 20 in the fully raised position, as illustrated in FIGS. 8, 9, and 12, has a height of approximately 49 inches and in the fully lowered position, as illustrated in FIGS. 1-5, and 11, and has a height of approximately 33 inches; and a first clearance 50 between the opposed telescoping vertical legs 28 and 30 under the table top 22 and within the spacing 42 of approximately 36.5 inches. These specific dimensions accomplish the invention as described herein and for proper weight and height distributions balance when the table 20 is in the fully lowered position and/or fully raised position.

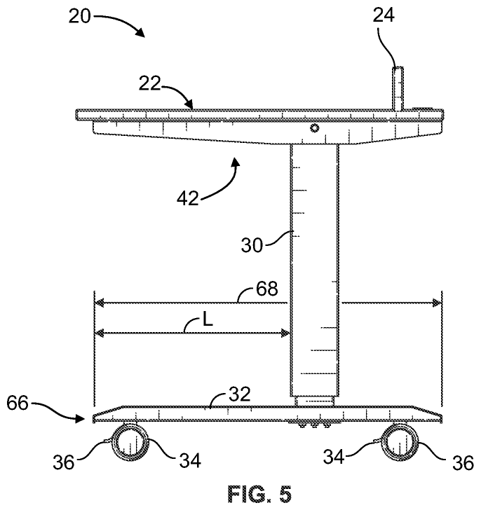

Additionally, each of the opposed telescoping vertical legs 28 and 30 are connected to the base 32 at a location L (see FIGS. 1 and 5), as measured from the front 66 of the base 32, which is also substantially the same distance from the front of the table 20. In the preferred embodiment, the location L is at a location that is more than or greater than 50% of the entire length 68 of the base 32. In this manner, this location L of the opposed telescoping vertical legs 28 and 30 defines the setback of each of the opposed telescoping vertical legs 28 and 30 from the front of the table 20 to further assist in the accommodation of the patient laying on the surgical chair 48 and use with the table 20.

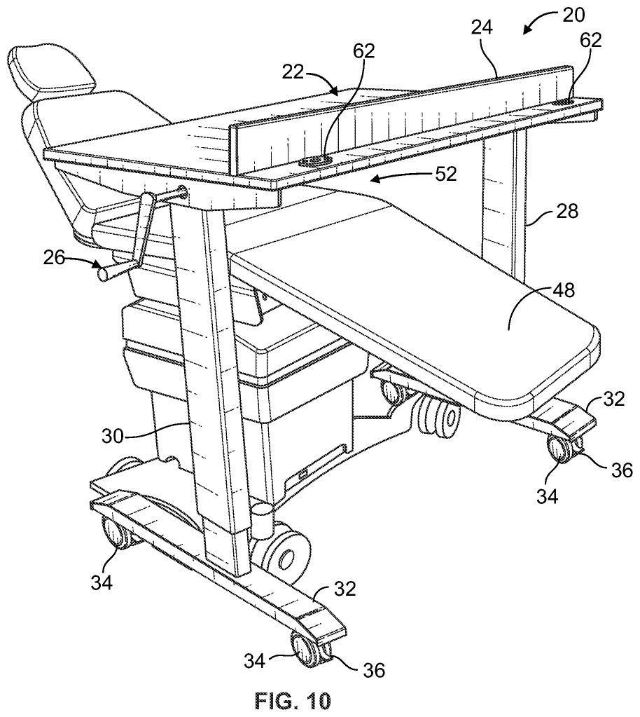

In use, the table 20 is rolled to a patient laying on a surgical chair or bed (collectively, “surgical chair”) 48, as illustrated in FIG. 10, the crank 26 is operated and adjustable to raise or lower the opposed telescoping vertical legs 28 and 30 to create a second clearance 52 between the first clearance 50 and within the spacing 42 between the table top 22 and the patient laying on the surgical chair 48. Once the table top 22 is at the proper height, the table 20 is rolled using the rolling coasters 34 such that the table top 22 is then properly moved or situated over the patient laying on the surgical chair 48 with the second clearance 52 providing the necessary spacing between the table top 22 and the patient. Once in place, the safety brakes 36 are engaged.

In this manner, the table top 22 is in the uniquely positioned to provide (i) a first clearance 50 between the opposed telescoping vertical legs 28 and 30 under the table top 22 and within the spacing 42 to accommodate the surgical chair or bed, (ii) a second clearance 52 within the spacing 42 between the table top 22 and the patient lying on the surgical chair or bed enabling the surgeon to use the table 20 situated over the patient to thereby perform surgery on the patient lying on the surgical chair or bed.

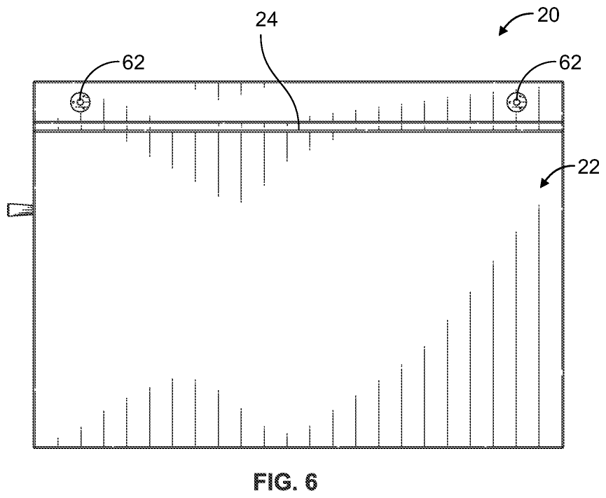

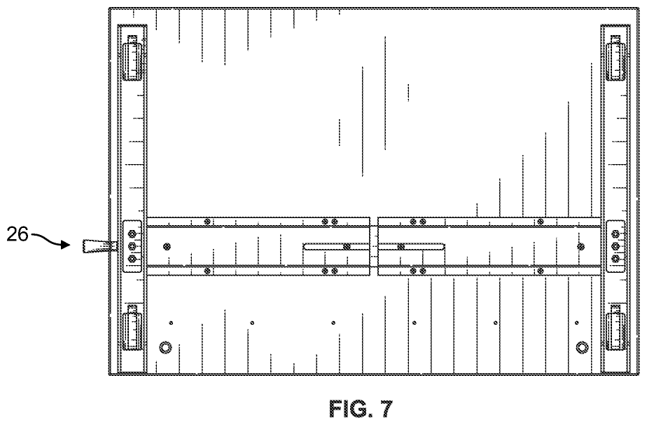

Additionally, a plurality of caps 62 (see FIGS. 6 and 10) can be removed to reveal holes therein which may then be used by the surgeon to attach or hang any additional surgical instruments or any other desired components.

Based on the foregoing method and apparatus for using Applicant's inventive table 20, Applicant's inventive table top 20 also provides the following advantages:
(a) the table 20 that uniquely solves the problem of providing surgical procedures to a patient lying on the surgical chair or bed;
(b) the table 20 that provides the specific clearances and dimensions to accommodate the patient lying on the surgical chair or bed which has never been done before; and
(c) the ability to then provide the patient with additional surgical package or tools necessary for the surgical procedure.

Thus, there has been provided Applicant's unique inventive over the patient adjustable surgical table in accordance with the present invention. While the invention has been described in conjunction with a specific embodiment, it is evident that many alternatives, modifications and variations will be apparent to those skilled in the art in light of the foregoing description. Accordingly, it in intended to embrace all such alternatives, modifications and variations as fall within the spirit and scope of the appended claims.

What is claimed is:

1. A table for use with a surgical chair, comprising:
a table top having a top side and a bottom side and defining opposed exterior edges separated by a length and a width between a front side and a back side;
a first telescoping leg situated adjacent to one of the opposed exterior sides of the bottom side of the table top;
a second telescoping leg situated adjacent to the other of the opposed exterior sides of the bottom side of the table top;
a first clearance provided between the first telescoping leg and the second telescoping leg;
a spacing created below the entirety of the bottom side of the table top and between the first telescoping leg and the second telescoping leg;
means for supporting each of the first telescoping leg and the second telescoping leg;
means for raising or lowering each of the first telescoping leg and the second telescoping leg;
wherein, upon using the means for raising or lowering each of the first telescoping leg and the second telescoping leg, the first telescoping leg and the second telescoping leg raise or lower the table top to increase or decrease the spacing for use with the surgical chair.

2. The table of claim 1 wherein the first clearance provided between the first telescoping leg and the second telescoping leg is less than the length.

3. The table of claim 1 wherein the means for supporting each of the first telescoping leg and the second telescoping leg is a first support base and a second support base, each of the first support base and the second support base having a base front side and a base back side.

4. The table of claim 3 wherein each of the first support base and the second support base have a base length that are substantially the same between the base front side and the base back side.

5. The table of claim 4 wherein the base length is substantially the same as the width of the table top.

6. The table of claim 5 wherein the first telescoping leg is affixed to the first support base at a location that is greater than fifty percent (50%) of the base length as measured from the base front side.

7. The table of claim 6 wherein the first telescoping leg is setback from the front side of the table top a distance that is substantially equal to the location.

8. The table of claim 7 wherein the second telescoping leg is affixed to the second support base at a location that is greater than fifty percent (50%) of the base length as measured from the base front side.

9. The table of claim 8 wherein the second telescoping leg is setback from the front side of the table top a distance that is substantially equal to the location.

10. A table for use with a surgical chair, comprising:
a table top having a top side and a bottom side and defining opposed exterior edges separated by a length and a width between a front side and a back side;
a first telescoping leg situated adjacent to one of the opposed exterior sides of the bottom side of the table top;
a second telescoping leg situated adjacent to the other of the opposed exterior sides of the bottom side of the table top;
a first clearance provided between the first telescoping leg and the second telescoping leg;
a spacing created below the entirety of the bottom side of the table top and between the first telescoping leg and the second telescoping leg;
a first support base supporting the first telescoping leg;
a second support base supporting the second telescoping leg;
means for raising or lowering each of the first telescoping leg and the second telescoping leg;
wherein, upon using the means for raising or lowering each of the first telescoping leg and the second telescoping leg, the first telescoping leg and the second telescoping leg raise or lower the table top to increase or decrease the spacing for use with the surgical chair.

11. The table of claim 10 wherein, in a fully raised position, the table top has a height of substantially forty-nine inches.

12. The table of claim 10 wherein, in fully lowered position, the table top has a height of substantially thirty-three inches.

13. The table of claim 10 wherein the first clearance between the first telescoping leg and the second telescoping leg is substantially thirty-six and one-half inches.

14. The table of claim 10 wherein the length of the table top between the opposed exterior edges is substantially the same as the first clearance.

15. The table of claim 10 wherein, when in use with a surgical chair, a second clearance is created between the first clearance and within the spacing between the table top and the surgical chair.
